(19) United States
(12) Patent Application Publication
(10) Pub. No.: US 2022/0111183 A1
(43) Pub. Date: Apr. 14, 2022

(54) Pulmonary Embolism Removal System

(71) Applicant: Terumo Corporation, Tokyo (JP)

(72) Inventor: Brian Gray, Aliso Viejo, CA (US)

(73) Assignee: Terumo Corporation, Tokyo (JP)

(21) Appl. No.: 17/499,711

(22) Filed: Oct. 12, 2021

Related U.S. Application Data
(60) Provisional application No. 63/090,630, filed on Oct. 12, 2020.

(51) Int. Cl. A61M 25/10; A61B 17/22

(52) U.S. Cl. CPC A61M 25/104; A61M 25/1011; A61B 17/22; A61B 2017/22079; A61B 2017/22048; A61B 2017/22067; A61M 2025/1043

(57) ABSTRACT

The system isolates a clot using a proximal obstruction device, such as a balloon, to stop blood flow. An expandable device is advanced through the clot and is expanded to disrupt the clot from the vessel walls. The expandable device may include the use of tines, a balloon, negative pressure, adhesives, or jet spray to disrupt and dislodge the clot.

### RELATED APPLICATIONS

This application claims benefit of and priority to U.S. Provisional Application Ser. No. 63/090,630 filed Oct. 12, 2020 entitled Pulmonary Embolism Removal System, which is hereby incorporated herein by reference in its entirety.

### BACKGROUND OF THE INVENTION

Pulmonary embolism (PE) is a manifestation of venous thromboembolism (VTE) most often associated with deep vein thrombosis (DVT). PE is a condition in which one or more blood vessels become blocked by a blood clot. The present invention is directed to a system and device for disrupting and removing these clots.

Current methods of clot removal include injectable medications that dissolve and disperse the clot, known as lytics; thrombectomy catheters; aspiration catheters; and mechanical methods to break up and extract the clot using balloons, stents, snares, or a combination thereof.

The introduction of mechanical devices into the body always presents the risk of causing trauma or irritation to the native tissues. Thus, devices can always be improved to be less traumatic. Additionally, removing clots or other tissue from a bloodstream carries with it the risk of the removed material being carried downstream in the circulatory system and causing further complications, e.g, stroke. Devices that perform such procedures should minimize, or ideally eliminate, this risk.

### OBJECTS AND SUMMARY OF THE INVENTION

The embodiments of the present invention are directed to catheter systems designed to remove blood clots from the pulmonary artery. These systems are designed to minimize irritation to the vessel as well as preventing the loss of material removed from the vessel.

One aspect of the invention provides a catheter system with distal and proximal balloons that are used to isolate a targeted pulmonary clot. A distal end of a suction catheter is located between the two balloons and is used to remove the clot material.

Another aspect of the invention provides a catheter system with distal and proximal balloons that are used to isolate a targeted pulmonary clot. A third balloon, or “membrane covered shaft” is advanceable through an access catheter that has a distal end located between the distal and proximal balloons. The membrane is a semipermeable membrane that can be inflated with a medicament or agent that seeps through the balloon to interact with the clot.

In one embodiment, the agent is an adhesive that binds to the clot, thereby adhering the clot to the membrane. Once adhered, the membrane covered shaft may be removed, removing the clot with the membrane. Additional similar or different tools may then be advanced through the access catheter to remove more material or conduct further procedures.

Another aspect of the invention is a catheter system that includes distal and proximal balloons for isolating a targeted pulmonary clot. The distal balloon is positioned on or distal of a spray nozzle having jet ports that are directed proximally. At a distal end of the proximal balloon, there is located a distal end of a suction catheter. Once positioned such that the distal and proximal balloons are on either side, and thus isolating, a clot, a liquid, or even a liquid agent, may be pumped through the proximally-directed jet ports to break up the clot while the material and liquid are being aspirated through the suction catheter.

In one embodiment, compressed CO2 gas may be delivered through the aforementioned jet ports to break up the clot allowing the suction catheter to remove clot material without removing excessive blood. The CO2 gas would then get absorbed into the body and be exhaled naturally.

Yet another aspect of the invention provides a catheter system that includes a proximal balloon for placement proximally of a targeted pulmonary clot, temporarily minimizing blood flow through the vessel and preventing migration of the clot. An expandable distal mechanism including tines or similar elements may then be expanded moved back and forth through the clot to mechanically break up and disengage the clot from the vessel walls. The proximal balloon terminates distally at the distal end of a catheter into which distal mechanism can be retracted to remove the clot.

Another aspect of the invention is a catheter system that includes a balloon guide catheter, an aspiration guide catheter and an expandable mechanism for macerating the clot. The macerator may include expandable tines that may be moved back and forth and/or rotated through the clot. Aspiration may be applied through the aspiration guide catheter during maceration of the clot. The balloon catheter occludes the blood supply during the maceration and aspiration.

Another aspect of the invention is a system for removing a clot from a vessel having a first catheter having a proximal end and a distal end and at least one lumen extending through the first catheter; a proximal balloon disposed around a distal portion of the first catheter; a second catheter having a proximal end and a distal end and being movable through a lumen in the first catheter; an expandable mechanism disposed at a distal region of the second catheter; and a clot retention mechanism disposed distal to the proximal balloon.

Another aspect of the invention is a method of removing clot material from a vessel that includes placing a first catheter to a location proximal to a clot; moving a second catheter through a lumen of the first catheter; penetrating a proximal end of the clot with the second catheter towards a distal end of the clot; blocking blood flow proximal of the clot with the first catheter; expanding a distal end of the second catheter; disrupting the clot; and, removing clot material of the disrupted clot through a lumen of the first catheter.

Another aspect of the invention is a device for removing a clot from a vessel comprising: a distal expandable device; a proximal expandable device; a clot disruption mechanism disposed distal to the proximal expandable device; wherein the proximal expandable device surrounds a catheter having an open distal end configured for removal of clot material from the vessel.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a side view of an embodiment of an embolism removal system of the invention;

FIG. 2 is a side view of the embodiment of FIG. 1 in an inflated state;

FIG. 3 is a side view of the embodiment of FIG. 1 during use;

FIG. 4 is a side view of an embodiment of an embolism removal system of the invention;

FIG. 5 is a view of a membrane component of the embodiment of FIG. 4;

FIG. 5A is a cross-sectional view of FIG. 5;

FIG. 6 is a side view of an embodiment of an embolism removal system of the invention;

FIG. 7 is a view of a distal end of the embodiment of FIG. 6;

FIG. 8 is a view of the embodiment of FIG. 6 in use;

FIG. 9 is a close-up perspective view of a portion of the embodiment of FIG. 6;

FIG. 10 is a perspective view of the embodiment of FIG. 6 in an inflated state;

FIG. 11 is a side view of a pump for use with embodiments of the invention;

FIG. 12 is a perspective view of a suction device for use embodiments of the invention;

FIG. 13 is a perspective view of an embodiment of an embolism removal system of the invention;

FIG. 14 is a perspective view of an embodiment of an embolism removal system of FIG. 13 in use;

FIG. 15 is a view of an embodiment of an embolism removal system of the invention;

FIG. 16 is a view of an aspiration guide catheter of an embolism removal system of the invention of FIG. 15;

FIG. 17 is a view of a partially deployed embolism removal system of the embodiment of FIG. 15;

FIG. 18 is a view of a partially deployed embolism removal system of the embodiment of FIG. 15;

FIG. 19 is a view of a deployed embolism removal system of the embodiment of FIG. 15;

FIG. 20 is a view of an embodiment of a distal tip of an embolism removal system of the invention;

FIG. 21 is a view of an embodiment of a distal tip of an embolism removal system of the invention;

FIG. 22 is a cross-sectional view of a distal tip of the embodiment of FIG. 21; and,

FIGS. 23A-23C are perspective views of further embodiments of an embolism removal system of the invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

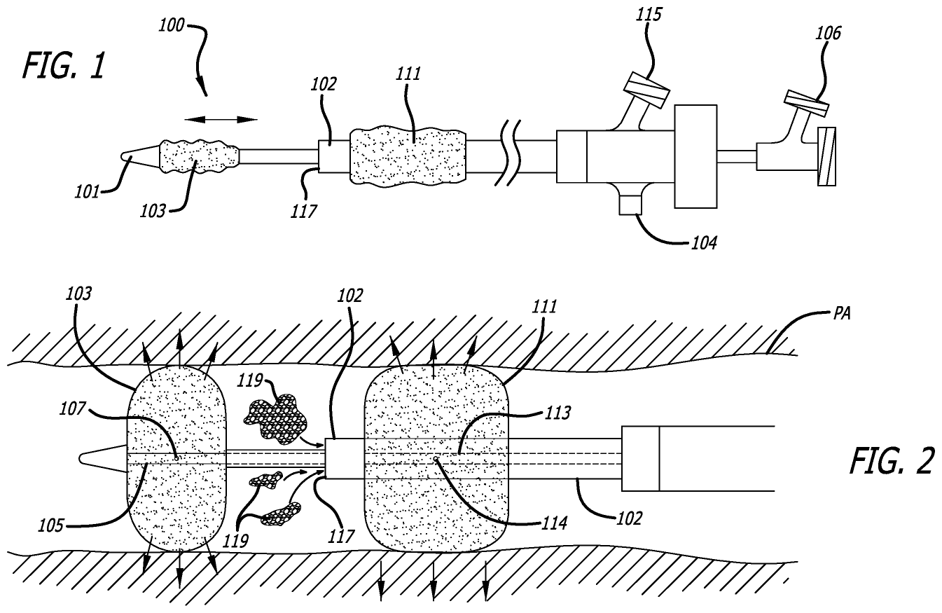

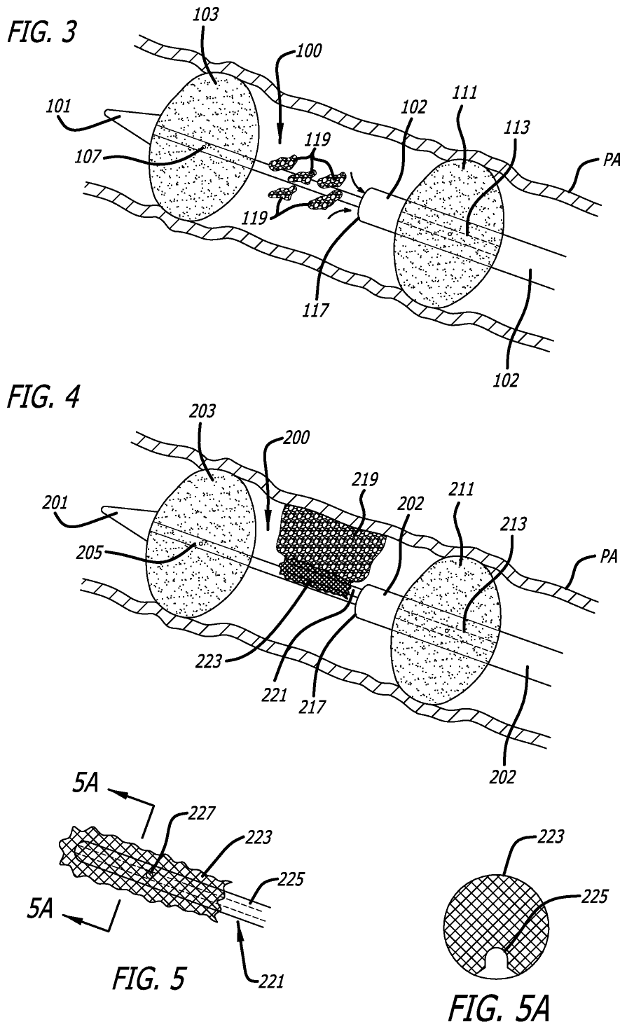

FIGS. 1-3 show an embodiment of a device for disrupting and removing an embolism and includes a balloon catheter 100 that is advanceable and retractable through a sheath 102. The balloon catheter 100 has a pointed distal tip 101 that may be advanced through a clot easily, minimizing the chances of breaking pieces of the clot loose. The balloon catheter 100 further includes a distal balloon 103 fed by an inflation lumen 105 that runs through the balloon catheter 100 to at least one distal fill port 106 at the proximal end of the device. A guidewire lumen (not shown) may also run through the balloon catheter 100 for use in navigation.

The sheath 102 has a distal end around which is placed a proximal balloon 111. The proximal balloon 111 is fed by an inflation port 114 and its inflation lumen 113 that runs along the length of the sheath 102 to a proximal fill port 115 at the proximal end of the device. The distal end of the sheath 102 has a second, larger lumen 117 through which the balloon catheter 100 passes and is connected at the proximal end of the device to an aspiration port 104. Thus, the larger lumen serves two purposes—aspiration and working channel.

Operation of the device of FIGS. 1-3 can be exemplified by the following: the sheath 102 is inserted through the femoral vein to the inferior vena cava (IVC), likely either over a guidewire or the device may be made to be steerable, through the right atrium and the right ventricle of the heart and into the targeted pulmonary artery PA. The balloon catheter 100 is then advanced through the clot 119 until the distal balloon 103 has cleared the clot 119 and is located distally thereof. The distal balloon 103 is then inflated with saline and/or contrast agent through the inflation lumen 105 and its inflation port 107. The proximal balloon 111 is also inflated similarly on a proximal side of the clot 119 through its inflation lumen 113 and inflation port 114. Doing so seals the vessel on either side of the clot 119.

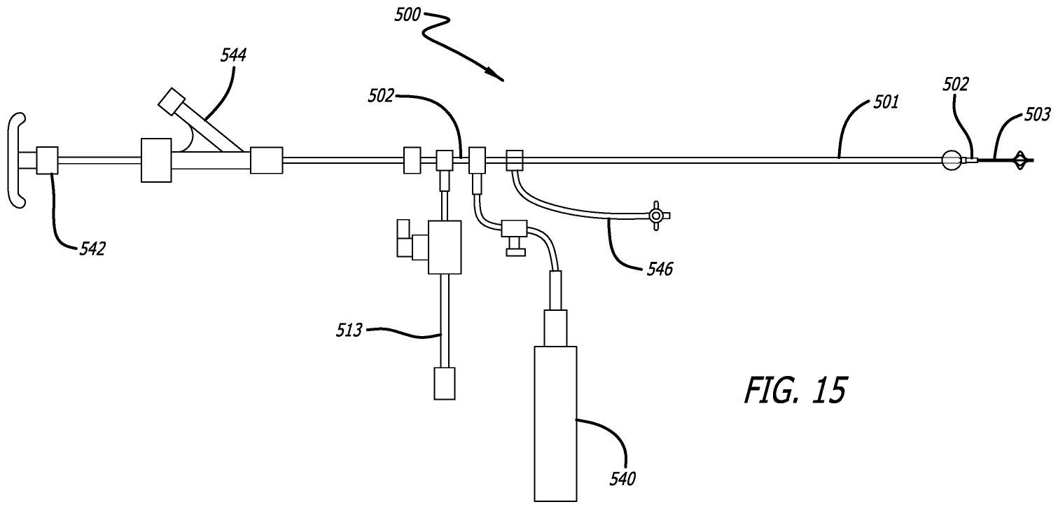

With the clot 119 sealed between the balloons 103, 111, a vacuum is drawn through the larger lumen 117 by attaching a pump (not shown) or vacuum syringe 540 (See FIG. 15) to the aspiration port. The vacuum causes the clot 119 to become disrupted e.g., dislodged, and removed from the pulmonary artery PA.

FIGS. 4-5 show an embodiment of a device for disrupting and removing an embolism and includes a balloon catheter 200 that is advanceable and retractable through a sheath 202. The balloon catheter 200 has a pointed distal tip 201 that may be advanced through a clot 219 easily, minimizing the chances of breaking pieces of the clot 219 loose. The balloon catheter 200 further includes a distal balloon 203 fed by an inflation lumen 205 that runs through the balloon catheter to a distal fill port at the proximal end of the device. A guidewire lumen may also run through the balloon catheter for use in navigation.

The sheath 202 has a distal end around which is placed a proximal balloon 211. The proximal balloon 211 is fed by an inflation lumen 213 that runs along the length of the sheath 202 to a proximal fill port at the proximal end of the device. The distal end of the sheath 202 has a second, larger lumen 217 through which the balloon catheter 200 passes. The larger lumen 217 is sized to accommodate a second catheter referred to herein as a removal catheter 221.

The shaft of the removal catheter 221 is covered with a membrane 223 at its distal end that forms a balloon-like device. Referring to FIGS. 5 and 5A, the membrane 223 is fed by a lumen 225 and port 227 that runs through the removal catheter 221. The membrane is semi-permeable such that when inflated through the lumen with an agent such as an adhesive, the agent permeates the membrane and interacts with the clot. The membrane 223 may be inflated by multiple lumens with ports on multiple sides of the removal catheter 221 to speed inflation and reduce resistance. This may also ensure faster, more uniform coverage of the membrane 223 with the agent, e.g, an adhesive.

One example of an agent for use with the device is N-butyl cyanoacrylate (NBCA), which is an adhesive that instantly binds to the clot 219. Once the clot 219 is bound to the membrane 223, the membrane 223 is then retracted and used to remove the clot 219.

Operation of the device of FIGS. 4-5A can be exemplified by the following: the sheath 202 is inserted through the femoral vein to the inferior vena cava (IVC), likely using a guidewire, or the device may be made to be steerable, through the right atrium and the right ventricle of the heart and into the targeted pulmonary artery PA. The balloon catheter 202 is then advanced through the clot 219 until the distal balloon 203 has cleared the clot 219 and is located distally thereof. The distal balloon 203 is then inflated with saline and/or contrast agent and the proximal balloon 211 is also inflated similarly on a proximal side of the clot 219. Doing so seals the vessel on either side of the clot 219.

With the clot 219 sealed between the balloons 203, 211, the removal catheter 221 is advanced out of the distal end of the sheath 202, bringing the membrane 223 in close proximity to the clot 219. An agent (not shown), such as NBCA is injected into the membrane 223, causing the membrane 223 to expand against the clot 219, while the agent seeps through the membrane 223 and disrupts the clot, e.g., causes the clot 219 to adhere to the membrane 223. The removal catheter 221 is then withdrawn from the sheath 202 and discarded, and a second removal catheter (not shown) may then be advanced to remove more of the clot 219, if necessary. The lumen 217 of the sheath 202 may alternatively be used to advance other tools, or may be connected to suction, if desired.

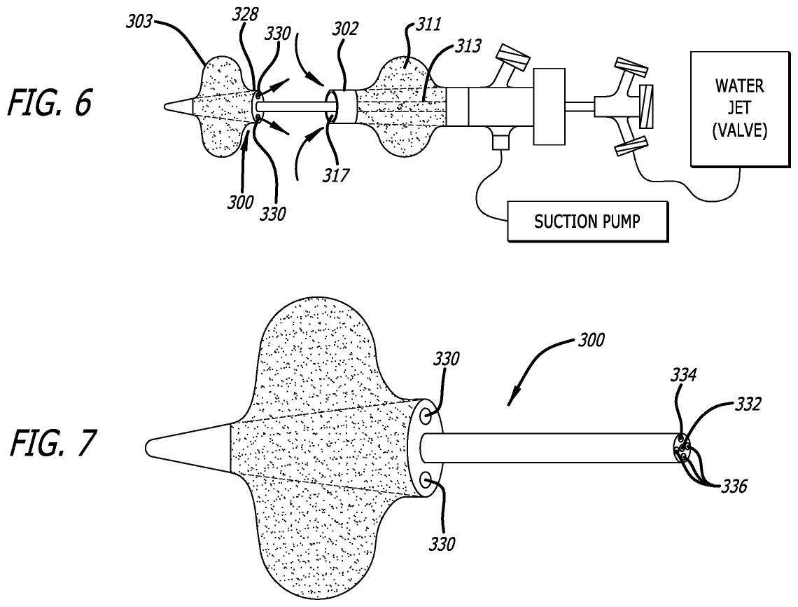

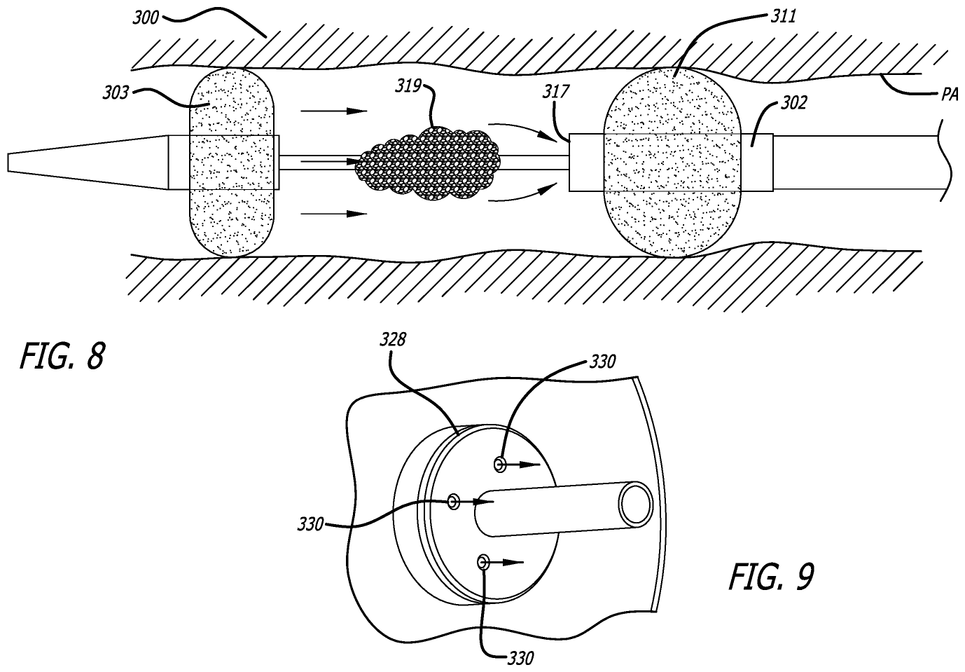

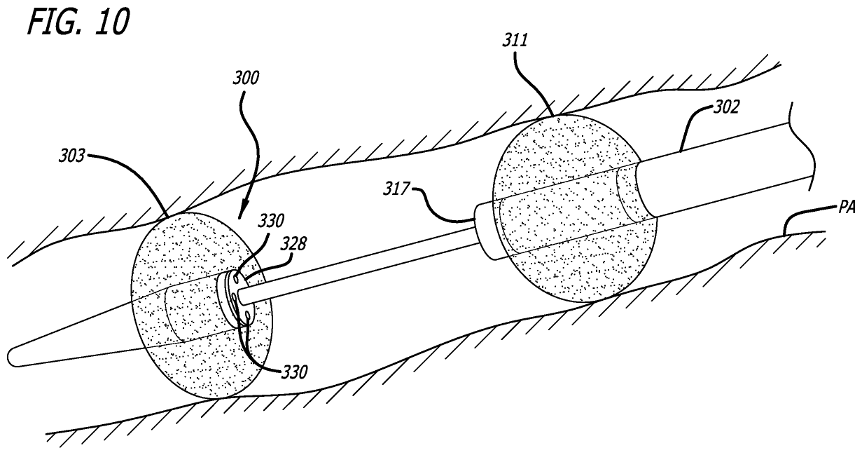

FIGS. 6-10 show an embodiment of a device for disrupting and removing an embolism and includes a balloon catheter 300 that is advanceable and retractable through a sheath 302. The balloon catheter 300 has a distal balloon 303 that is positioned on or distal of a spray nozzle 328 having jet ports 330 that are directed proximally. The distal balloon 303 is fed by an inflation lumen 334 that runs through the balloon catheter 300 to a distal fill port at the proximal end of the device in a manner as disclosed in previous embodiments. A second lumen 336 (or multiple lumens 336) is used to deliver pressurized fluid to the jet ports 330. The fluid may be saline, an agent, or a combination thereof. A guidewire lumen 332 may also run through the balloon catheter 300 for use in navigation. FIG. 7 shows a close up view of the distal balloon catheter 300 and an embodiment of the configuration of the lumens 332, 334, 336.

The sheath 302 has a distal end around which is placed a proximal balloon 311. The proximal balloon 311 is fed by an inflation lumen 313 that runs along the length of the sheath to a proximal fill port at the proximal end of the device. The distal end of the sheath has a second, larger lumen 317 through which the balloon catheter 300 passes and is connected at the proximal end of the device to an aspiration port. Thus, the larger lumen 317 serves two purposes—aspiration and working channel.

Operation of the device of FIGS. 6-10 can be exemplified by the following: the sheath is inserted through the femoral vein to the inferior vena cava (IVC), likely via a guidewire, or the device may be made to be steerable, through the right atrium and the right ventricle of the heart and into the targeted pulmonary artery PA. The balloon catheter 300 is then advanced through the clot 319 until the distal balloon 303 has cleared the clot 319 and is located distally thereof. The distal balloon 303 is then inflated with saline and/or contrast agent and the proximal balloon 311 is also inflated similarly on a proximal side of the clot 319. Doing so seals the vessel on either side of the clot.

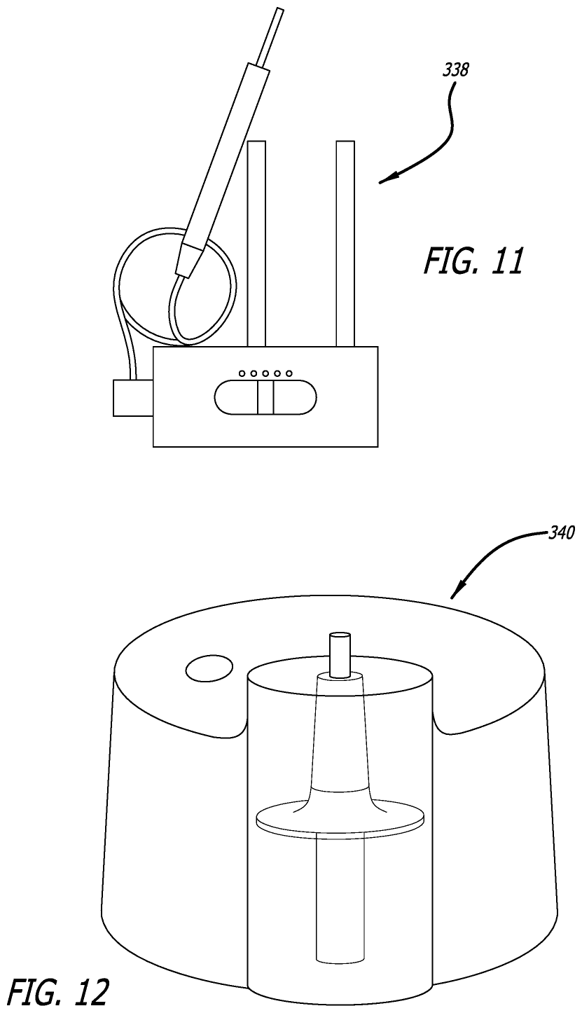

With the clot sealed between the balloons 303, 311, pressurized fluid is delivered through the jet ports 330, creating fluid streams that are powerful enough to disrupt, e.g., dislodge, the clot 319. A vacuum is drawn through the larger lumen 317 by attaching a pump 338 (FIG. 11) to the aspiration port. The vacuum acts in conjunction with the jets to dislodge and remove the clot 319 from the pulmonary artery PA.

Alternatively, the device of FIGS. 6-10 can be used to deliver compressed CO2 gas through the jet ports 330 in order to dislodge the clot 319. The gas would either be aspirated through the vacuum lumen 317 or be absorbed by the blood stream and exhaled through the lungs. The gas may be used to remove the clot 319 while displacing the blood out of the chamber created between the two balloons 303, 311 prior to applying a vacuum to the suction catheter 317. This way the clot will be removed by the suction catheter 317 without removing healthy blood. CO2 is easily and naturally absorbed into the bloodstream.

FIG. 11 shows a positive pressure pump 338 that can be used with the invention, in particular, with the jet nozzle embodiment of FIGS. 6-10. Various embodiments of positive displacement and non-positive displacement pumps could be configured for use with the embodiments of the invention.

FIG. 12 shows an embodiment of a negative pressure (suction) pump 340 that may be used for aspiration with the various embodiments of the invention. In one embodiment, a pump such as the Gomco™ Aspirator Pump, Model 405 (manufactured by Allied Healthcare Products, Inc. St. Louis, Mo.) as is known in the art, may be used. In one embodiment, the pump has the ability to control vacuum pressures up to 635 mm Hg with a flow rate of 40 Liter/minute at open flow. In one embodiment, the pump is used on conjunction with a disposable 1.5 Liter collection canister.

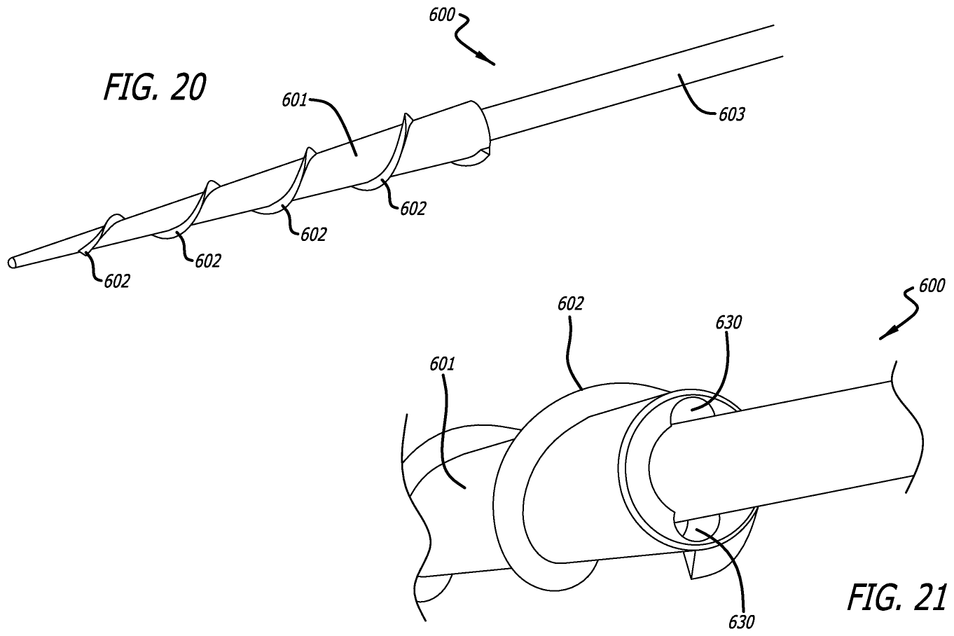

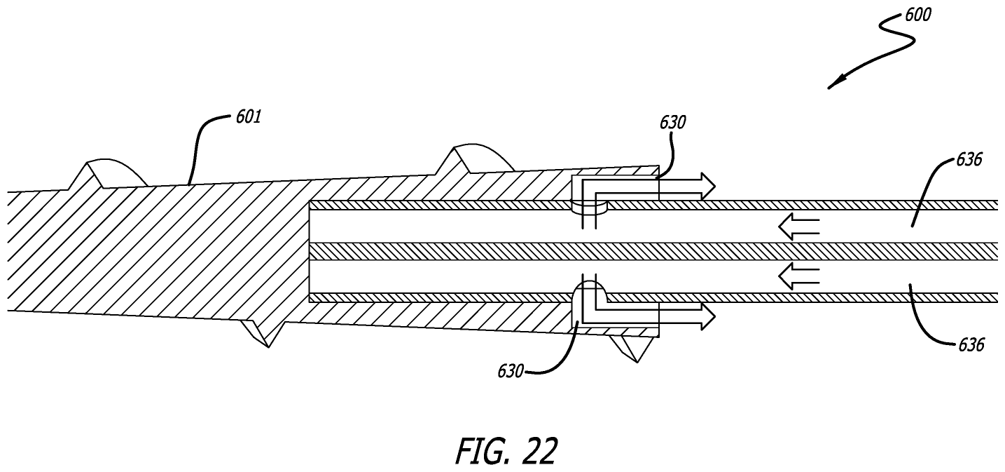

FIGS. 20-22 show an embodiment similar to the embodiments of FIGS. 6-10. It includes a catheter device 600 having a pointed distal tip 601 that has screw-like features 602, e.g., threads for penetrating a clot. The catheter 600 also includes a shaft 603 connected to the distal tip 601.

In a manner similar to the embodiment disclosed in FIG. 7, the shaft 603 of the catheter 600 includes one or more lumens 636 for directing pressurized fluid to the pointed distal tip 601, which pressurized fluid is then ejected towards the clot through jet ports 630. The operation of the embodiment of FIGS. 20-22 is analogous to that explained above with reference to FIGS. 6-10.

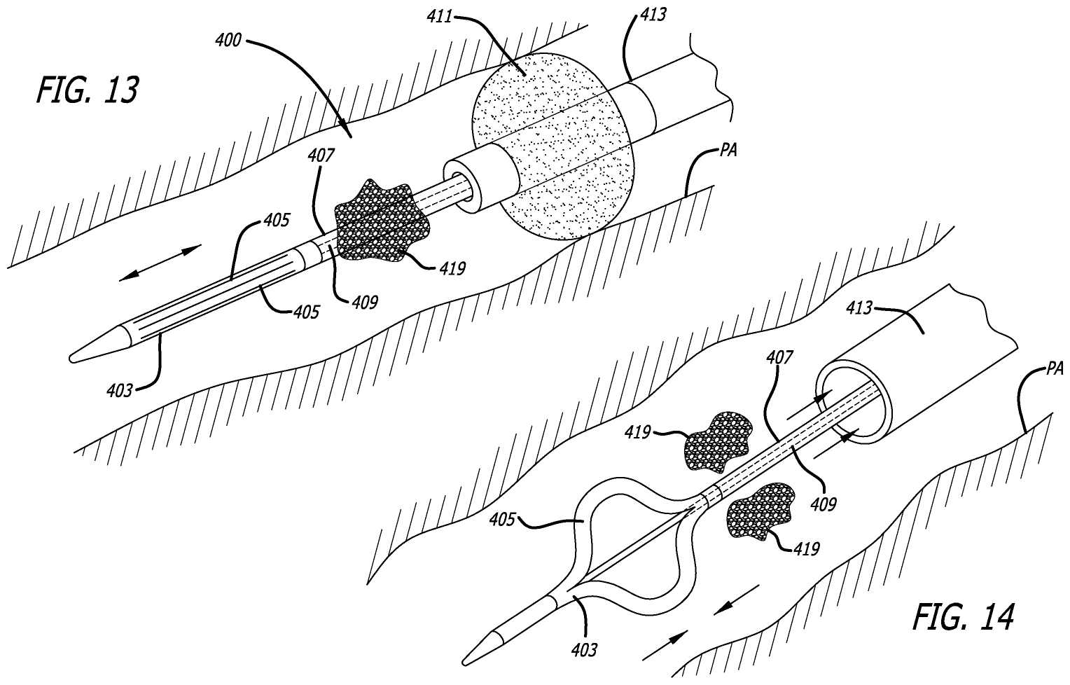

FIGS. 13-14 show an embodiment of a device 400 for disrupting and removing an embolism and includes an expandable distal mechanism 403 that has tines 405 or similar elements designed to be expanded and moved back and forth through the clot 419 to mechanically break up and disengage the clot 419 from the vessel walls. The expandable distal mechanism 403 is attached to an inner catheter 407 having a lumen that carries a push rod 409. The distal end of the mechanism 403 is connected to the distal end of the push rod 409 and the proximal end of the mechanism is attached to the distal end of the inner catheter 407. By pulling the push rod proximally relative to the inner catheter, the distance between the distal and proximal ends of the mechanism 403 shortens, causing the mechanism 403 to flare and expand. In this expanded state, the inner catheter and the push rod may be advanced and retracted in unison in order to push and pull the expanded mechanism through a clot 419, thereby dislodging the clot 419.

The embodiment also includes a proximal balloon 411 that terminates distally at the distal end of a sheath catheter 413 into which the inner catheter 407, push rod 409, and expandable mechanism 403 can be retracted to remove the clot. The proximal balloon 411 is fed by an inflation lumen that runs along the length of the sheath to a proximal fill port at the proximal end of the device.

Operation of the device of FIGS. 13-14 can be exemplified by the following: the sheath catheter 413 is inserted over a guidewire, or is steerable, through the femoral vein to the inferior vena cava (IVC), through the right atrium and the right ventricle of the heart and into the targeted pulmonary artery PA. The proximal balloon 411 is then inflated with saline and/or contrast agent, thus temporarily stopping the blood flow through the vessel. The inner catheter 407 is then advanced through the clot 419 until the expandable mechanism 403 has cleared the clot 419 and is located distally thereof.

Next, the push rod 409 is retracted while holding the inner catheter 407 in place such that the expandable mechanism 403 expands. The push rod 409 is fixed relative to the inner catheter 407 and the two are pulled through the clot 419 in order to dislodge the clot 419 from the vessel walls. The expandable mechanism is pulled into the sheath catheter with the clot. The push rod may be advanced slowly relative to the inner catheter in order to ease retraction of the expandable mechanism into the sheath catheter coaxially.

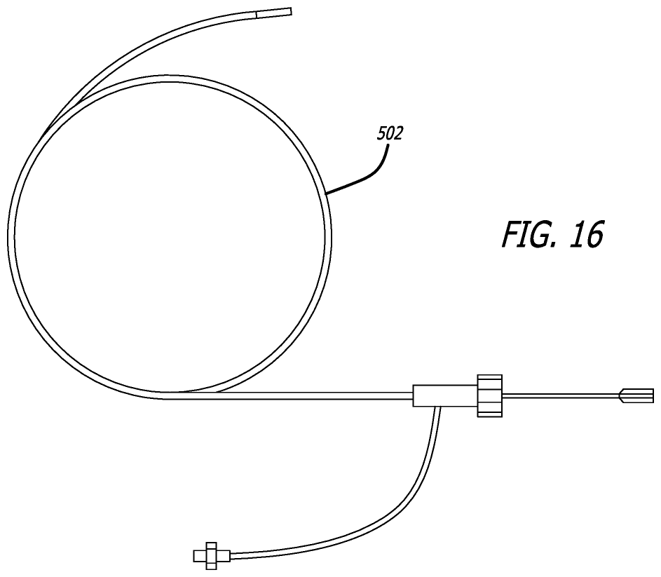

FIGS. 15-19 show an embodiment of a system 500 for disrupting and removing an embolism and includes a balloon guide catheter 501, and aspiration guide catheter 502 and an expandable mechanism 503 that serves as a clot macerator.

The expandable mechanism 503 is analogous to the expandable mechanism 403 of FIGS. 13-14 and includes tines 505 or similar elements designed to be expanded and moved back and forth and or rotated through the clot to mechanically break up and disengage the clot from the vessel walls. The expandable distal mechanism 503 is attached to an inner catheter 507 having a lumen that carries a push rod 509. A handle 542 is connected to the push rod 509. The distal end of the mechanism 503 is connected to the distal end of the push rod 509 and the proximal end of the mechanism is attached to the distal end of the inner catheter 507. By pulling on the handle 542, the push rod moves proximally relative to the inner catheter and the distance between the distal and proximal ends of the mechanism 503 shortens, causing the mechanism 503 and its associated tines 505 to flare and expand. In this expanded state, the inner catheter 507 and the push rod 509 may be advanced and retracted and/or rotated in unison in order to push and pull and or rotate the expanded mechanism 503 through a clot, thereby disrupting, e.g., dislodging, the clot.

In one embodiment, the number of times 505 is four. However, more or less tines are possible depending on clot size and or hardness.

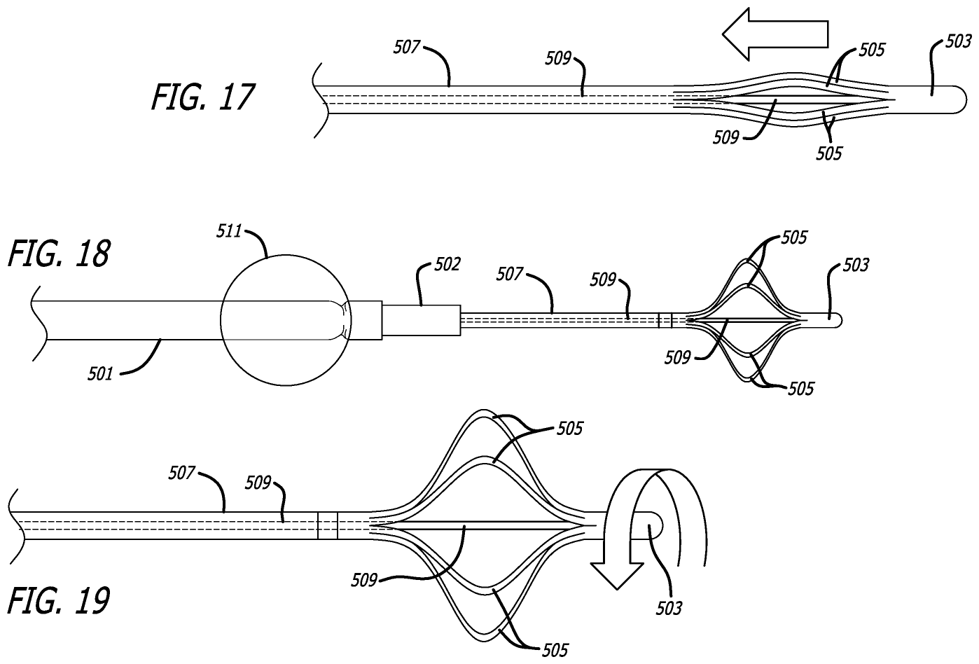

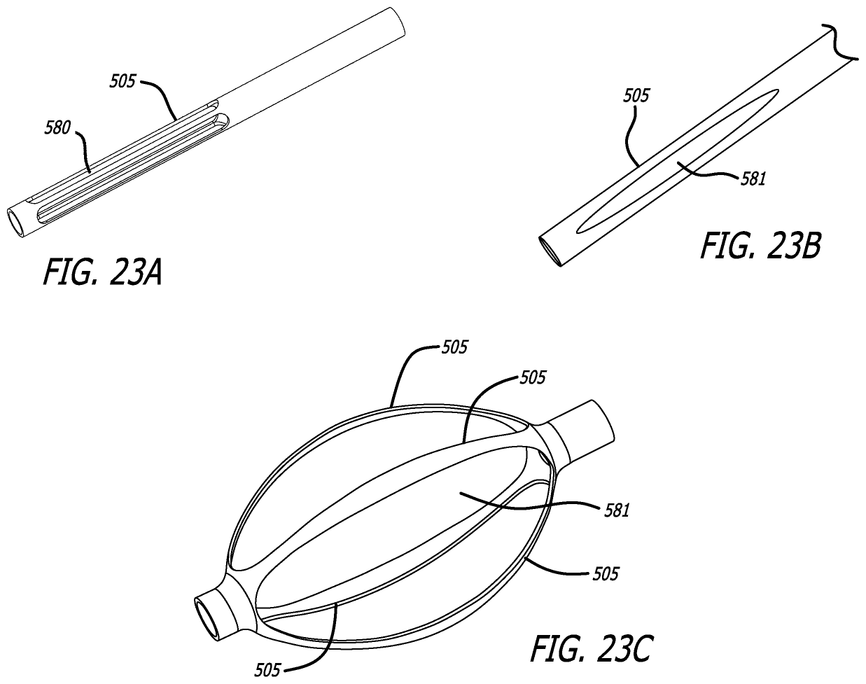

In one embodiment the shape of the tines 505 in an unexpanded state are separated by an elongated oval space 580 between the tines 505 as seen in FIG. 23A. The expanded shape of this embodiment is depicted in FIGS. 17-19.

In one embodiment the shape of the tines 505 in an unexpanded state are separated by a “cateye”-like or oblong oval shape 581 as seen in FIG. 23B. The expanded shape of this embodiment is depicted in FIG. 23C.

In one embodiment, the shape of the tines is a shape that requires a low and uniform force to expand the tines 505.

In one embodiment, the tines 505 are laser cut from a hypotube comprised of a Nitinol alloy. In another embodiment, the tines 505 are a braided cable.

The embodiment also includes a proximal balloon 511 that terminates distally at the distal end of the balloon guide catheter 501. The balloon is inflatable through a balloon inflation port 546. The aspiration guide catheter 502 extends distally from within the balloon guide catheter 501 and houses a lumen to which a negative pressure or suction pump 340 (FIG. 12) or vacuum syringe 540 (FIG. 15) is attached for applying suction to the clot. Extending out from the aspiration guide catheter 502 is the expandable mechanism 503, which functions as discussed above. The proximal balloon 511 is fed by an inflation lumen that runs along the length of the balloon guide catheter 501 to a proximal fill port at the proximal end of the device.

Although the embodiment of FIGS. 15-19 contemplates three catheters, it is noted that the balloon guide catheter 501 can be used independently of the aspiration guide catheter 502 and vice versa. In other words, each of the balloon guide catheter 501 and the aspiration guide catheter 502 can be independently used to provide an aspiration function. Similarly, each of the balloon guide catheter 501 and aspiration guide catheter 502 can be used independently with the expandable mechanism 503.

Operation of the device of FIGS. 15-19 can be exemplified by the following: The balloon guide catheter 501 is inserted over a guidewire, or is steerable, through the femoral vein to the inferior vena cava (IVC), through the right atrium and the right ventricle of the heart and into the targeted pulmonary artery PA. The proximal balloon 511 is then inflated with saline and/or contrast agent, thus temporarily stopping the blood flow through the vessel. The aspiration guide catheter 502 is then advanced through the balloon guide catheter 501 to a position proximal to the clot. The expandable mechanism or clot macerator is then advanced through the aspiration guide catheter 502 until the expandable mechanism 503 has cleared the clot and is located distally thereof.

Next, the push rod 509 is retracted while holding the inner catheter 507 in place such that tines 505 of the expandable mechanism 503 expand. In this expanded state, the inner catheter 507 and the push rod 509 may be advanced and retracted and/or rotated in unison in order to push and pull and or rotate the expanded mechanism 503 through a clot, thereby dislodging the clot. Simultaneously, negative pressure or suction may be applied through the aspiration guide catheter 502. The expandable mechanism 503 may then be pulled into the aspiration guide catheter 502 with the clot. The push rod 509 may be advanced slowly relative to the aspiration guide catheter 502 in order to ease retraction of the expandable mechanism into the aspiration guide catheter 502.

In one embodiment, saline is injected through flush port 544. The flush port 544 is in fluid communication with the space between the inner catheter 507 and the push rod of the expandable mechanism 503. The injection of saline purges air from the space between the inner catheter 507 and the push rod 509 and may be performed prior to conducting clot disruption, e.g., clot maceration.

Further embodiments include operation of the balloon guide catheter 501 independent of the aspiration guide catheter 502 with or without use of the expandable mechanism 503. In this regard, suction of the clot can be achieved with negative pressure using suction pump 340 or vacuum syringe 540. In yet a further embodiment, the aspiration guide catheter 502 can be used apart from the balloon guide catheter 501 along with the expandable mechanism 503 in conjunction with suction pump 340 or vacuum syringe 540 attached to proximal aspiration port 513.

It should be appreciated from the disclosed embodiments above that a clot retention mechanism or a clot disruption mechanism may be constituted, for example, by mechanisms of suction, vacuum, the application of adhesives, the application of jets of fluid in the form of a liquid or gas and the expansion of tines. It is also appreciated that in some embodiments those mechanisms can be used alone or in various combinations with each other. For example, suction can be used alone in combination with the application of adhesives, the application of jets of fluid or the expansion of times.

It is also appreciated that the clot retention mechanism or the clot disruption mechanism can simultaneously serve as an expandable mechanism as discussed in the embodiments above. For example, in one embodiment of FIGS. 13-19, the tines 405 (FIGS. 13-14) and tines 505 (FIGS. 15-19) constitute both an expandable mechanism as well as the clot retention mechanism or clot disruption mechanism.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A system for removing a clot from a vessel comprising:
a first catheter having a proximal end and a distal end and at least one lumen extending through the first catheter;
a proximal balloon disposed around a distal portion of the first catheter;
a second catheter having a proximal end and a distal end and being movable through a lumen in the first catheter;
an expandable mechanism disposed at a distal region of the second catheter;
a clot retention mechanism disposed distal of the proximal balloon.

2. The system of claim 1 wherein the expandable mechanism comprises a balloon.

3. The system of claim 1 wherein the expandable mechanism comprises expandable tines.

4. The system of claim 1 wherein the clot retention mechanism comprises a vacuum.

5. The system of claim 1 wherein the clot retention mechanism comprises a semi-permeable membrane containing an adhesive.

6. The system of claim 1 wherein the clot retention mechanism comprises a pressurized fluid.

7. The system of claim 1, further comprising a third catheter having a proximal end and a distal end and movable through a lumen of the first catheter; the third catheter being an aspiration catheter.

8. A method of removing clot material from a vessel comprising:
placing a first catheter to a location proximal to a clot;
moving a second catheter through a lumen of the first catheter;
penetrating a proximal end of the clot with the second catheter towards a distal end of the clot;
blocking blood flow proximal of the clot with the first catheter;
expanding a distal end of the second catheter;
disrupting the clot;
removing clot material of the disrupted clot through a lumen of the first catheter.

9. The method of claim 8 wherein expanding a distal end of the second catheter comprises sealing the vessel distal of the clot.

10. The method of claim 9 wherein disrupting the clot comprises applying suction to the clot.

11. The method of claim 8 wherein expanding a distal end of the second catheter comprises expanding a plurality of tines.

12. The method of claim 8 wherein expanding a distal end of the second catheter comprises expanding a balloon.

13. The method of claim 8 wherein disrupting the clot comprises adhering the clot to a mechanical component.

14. The method of claim 8 wherein removing clot material comprises mechanically removing the clot material from the vessel.

15. The method of claim 8 wherein removing clot material comprises applying a vacuum to a lumen of the first catheter.

16. The method of claim 8 wherein removing the clot material comprises mechanically pulling the clot material into a lumen of the first catheter.

17. The method of claim 8 wherein disrupting the clot comprises directing a jet of liquid onto the clot.

18. The method of claim 8 wherein disrupting the clot from the vessel comprises directing a jet of gas onto the clot.

19. The method of claim 8, further comprising moving a third catheter through the first catheter to a location proximal to the clot prior to moving the second catheter.

20. The method of claim 19, wherein removing the clot material comprises applying suction to the clot material through the third catheter.

21-25. (canceled)
